(19) United States
(12) Patent Application Publication
(10) Pub. No.: US 2025/0263660 A1
(43) Pub. Date: Aug. 21, 2025

(54) METHOD FOR IN VITRO EXPANSION OF CRYOPRESERVED CORD BLOOD-DERIVED REGULATORY T CELLS (Tregs) WITH HIGH RECOVERY RATE

(71) Applicant: CENTRAL SOUTH UNIVERSITY, Changsha (CN)

(72) Inventors: Zhiguang ZHOU, Changsha (CN); Haibo YU, Changsha (CN)

(73) Assignee: CENTRAL SOUTH UNIVERSITY, Changsha (CN)

(21) Appl. No.: 18/992,204

(22) PCT Filed: Jul. 6, 2023
(86) PCT No.: PCT/CN2023/106036

§ 371 Date: Jan. 8, 2025

(30) Foreign Application Priority Data
Jul. 8, 2022 (CN) 202210805505.X

(51) Int. Cl. C12N 5/0783; C12N 5/00

(52) U.S. Cl. CPC C12N 5/0637; C12N 5/525; C12N 5/562; C12N 2500/90

(57) ABSTRACT

A method for in vitro expansion of cryopreserved cord blood-derived regulatory T cells (Tregs) with a high recovery rate is provided, including the following steps: recovering the cryopreserved cord blood-derived Tregs after first expansion, and resuspending the cells with a serum-free medium; and adding a resulting suspension to an expansion culture medium, and conducting a second expansion culture, where a primary culture is conducted for 1 d to 2 d, then a subculture is conducted once every 1 d to 3 d, and a total culture time is 13 d or more. In the expansion culture medium, interleukin-2 (IL-2) is further added. The method can achieve the second recovery and expansion of cryopreserved Tregs produced after the first expansion. Tregs produced after the second recovery and expansion can have a viability of 90% or more, and can be further expanded (such as third expansion and fourth expansion).

### TECHNICAL FIELD

The present disclosure belongs to the field of cell therapy, and in particular relates to a method for in vitro expansion of cryopreserved cord blood-derived regulatory T cells (Tregs) with a high recovery rate.

### BACKGROUND TECHNOLOGY

Immune diseases involve the impaired functions of various organs and tissues caused by autoimmune disorders in patients, and have a wide disease spectrum, including autoimmune diabetes, systemic lupus erythematosus, etc. Autoimmune diseases are also listed as one of the ten major diseases in the Outline of the National Program for Medium- and Long-term Science and Technology Development of China. China has a large population base and a large absolute number of patients. Immune diseases not only impose a heavy economic burden on individuals and families, but also cause a serious impact on the economic development in China. The current treatments for immune diseases mostly rely on the traditional methods such as immunosuppressants. However, the immunomodulatory disorders as a main cause for immune diseases have not been solved.

Regulatory T cells (Tregs) are a special T cell subset that maintains the dynamic immune homeostasis by inhibiting and regulating effector T cells. Since the isolation and identification of Tregs by Japanese scholars at the end of the last century, Tregs have become a hot topic in the research of immunity in recent years. A variety of clinical trials related to Tregs have been initiated outside China. The representative Treg therapy for graft versus host disease has confirmed the safety and efficacy of Treg therapy in treating immune diseases. However, Treg therapy for immune diseases is still blank in China. Treg therapy can restore the balance of the immune system to prevent the further damage of autoimmune responses to organs and tissues and improve or even reverse a disease state, thereby fundamentally curing an immune disease.

More and more studies have shown that the cord blood is rich in Tregs, which are mostly Naïve T cells because these Tregs are not activated by antigens. Tregs expanded in vitro have excellent functions and low immunogenicity, which brings new hope for the treatment of immune diseases. At an early stage, the team of the present disclosure has established an in vitro isolation, expansion, and functional identification method for cord blood-derived Tregs that was approved as a national invention patent (patent No.: 201710613737.4), has carried out the clinical study of treating autoimmune diabetes with cord blood-derived Tregs for the first time internationally, and has preliminarily clarified the safety and efficacy of cord blood-derived Tregs in the treatment of autoimmune diabetes. In 2019 and 2020, the Juvenile Diabetes Cure Alliance (JDCA) published the annual reports on the global technologies for treating type 1 diabetes, respectively. The “cord blood-derived Treg” therapy technology has continuously been rated as the leading technology worldwide. Globally, there is currently no clinical trial for using cord blood-derived Tregs to treat an immune disease. Tregs can be extracted from the cord blood, expanded, and then used, which takes 14 d to 21 d. Thus, the clinical on-demand access requirement cannot be met. When the cryopreserved Tregs are recovered and then expanded, the cells will die in large quantities, which seriously affects the clinical use of Tregs. Therefore, it is urgent to establish an optimized method for in vitro expansion of cryopreserved Tregs with a high recovery rate and establish a Treg bank, thereby meeting the needs of clinical patients.

### CONTENT OF THE INVENTION

A technical problem to be solved by the present disclosure: A method for in vitro expansion of cryopreserved cord blood-derived Tregs with a high recovery rate is provided to overcome the deficiencies and shortcomings mentioned in the above background.

In order to solve the above technical problem, the present disclosure provides the following technical solutions:

A method for in vitro expansion of cryopreserved cord blood-derived Tregs with a high recovery rate is provided, including the following steps:
(1) recovering the cryopreserved cord blood-derived Tregs after first expansion, and resuspending the cells with a serum-free medium to produce a suspension; and
(2) adding the suspension to an expansion culture medium, and conducting a second expansion culture, where a primary culture is conducted for 1 d to 2 d, then a subculture is conducted once every 1 d to 3 d, and a total culture time is 13 d or more,
where in volume fractions, the expansion culture medium includes 70.54% to 85.27% of a serum-free medium, 2.5% to 5% of a 4-hydroxyethylpiperazine ethanesulfonic acid buffer, 1% to 2% of a penicillin-streptomycin solution, 1% to 2% of L-glutamine, 50 mol/L to 100 mol/L of 2-mercaptoethanol, 0 U/mL to 1,000 U/mL of recombinant interleukin-2 (IL-2), 50 nmol/L to 200 nmol/L of rapamycin, and 10% to 20% of AB serum.

In the method for in vitro expansion of cryopreserved cord blood-derived Tregs with a high recovery rate, preferably, a process of producing the cryopreserved cord blood-derived Tregs after the first expansion includes the following steps: preparing Tregs, adding an expansion culture medium to Tregs carrying magnetic beads, and conducting a first expansion culture, where a primary culture is conducted for 1 d to 2 d, then a subculture is conducted once every 1 d to 3 d, and a total culture time is 18 d or more.

Preferably, a process for the preparing Tregs includes the following steps:
S1, isolating a peripheral blood mononuclear cell (PBMC) layer from cord blood, removing red blood cells with a red blood cell lysis buffer, washing, and counting; conducting centrifugation, removing a resulting supernatant, and cryopreserving; and recovering and counting;
S2, conducting centrifugation, removing a resulting supernatant, and resuspending cells with an Easy Buffer to produce 0.5 mL to 6 mL of a suspension with a cell concentration of 5×10⁷ cells/mL; and
S3, transferring the cord blood-derived Treg-containing suspension into a sterile tube matching with a magnetic pole, adding CD25 Positive Selection Cocktail, and incubating for 5 min; vortexing Releasable Rapidsphere for 30 s or more until magnetic bead aggregates disappear; adding the Releasable Rapidsphere; adding a CD4⁺ T cell enrichment antibody mixture, and incubating for 5 min; adding an Easy Buffer to 10 mL, and gently mixing 2 times to 3 times; placing the sterile tube on the magnetic pole, and incubating for 10 min; preparing a centrifuge tube to collect CD25⁻ cells, and pouring a liquid in the sterile tube into the centrifuge tube under the magnetic pole; removing the sterile tube from the magnetic pole, adding 10 mL of an Easy Buffer to the sterile tube, gently mixing 2 times to 3 times, placing the sterile tube on the magnetic pole, and incubating for 5 min; adding an Easy Buffer to the initial resuspension volume with all cells on a tube wall rinsed off, adding a magnetic bead-removing buffer; adding a CD127^{high}-removing antibody mixture, and incubating for 5 min; adding an Easy Buffer to 10 mL, and thoroughly mixing 2 times to 3 times; placing the sterile tube on the magnetic pole, and incubating for 5 min; and preparing a centrifuge tube to collect Tregs, and pouring a liquid in the sterile tube into the prepared centrifuge tube under the magnetic pole.

More preferably, an amount of the CD25 Positive Selection Cocktail added is the resuspension volume×50 μL, an amount of the Releasable Rapidsphere added is the resuspension volume×30 μL, an amount of the CD4⁺ T cell enrichment antibody mixture added is the resuspension volume×50 μL, an amount of the magnetic bead-removing buffer added is the resuspension volume×100 μL, and an amount of the CD127^{high}-removing antibody mixture added is the resuspension volume×50 L.

In the method for in vitro expansion of cryopreserved cord blood-derived Tregs with a high recovery rate, preferably, in the expansion culture, cells are added to a 48-well plate at a concentration of 2×10⁵ to 8×10⁵/mL per well, and 0.5 mL of the expansion culture medium is added to each well for culture.

Preferably, both the primary culture and the subculture are conducted at 36° C. to 38° C. and 4% to 6% CO₂.

Preferably, on day 1 to day 2 after inoculation, 0.5 mL to 1 mL of the expansion culture medium and 100 U/mL to 400 U/mL of rhIL-2 are added to cells in each well, and then a culture is conducted.

More preferably, 0.5 mL of a medium in each well is removed, the remaining medium is thoroughly mixed with cells, cells in each well are passaged to 2 wells, and the medium is supplemented to 1 mL per well; and when the subculture is conducted a third time, the anti-CD3CD28 magnetic beads are removed, and then fresh anti-CD3CD28 magnetic beads are added with a number ratio of the magnetic beads to Tregs being 1:1.

In the method for in vitro expansion of cryopreserved cord blood-derived Tregs with a high recovery rate, preferably, in the expansion culture medium, IL-2 is further added; and more preferably, a concentration of the IL-2 in the expansion culture medium is 10 U/mL to 1,000 U/mL and preferably 200 U/mL to 600 U/mL.

Compared with the prior art, the present disclosure has the following beneficial effects:

1. The method of the present disclosure can achieve the second recovery and expansion of cryopreserved Tregs produced after the first expansion. Tregs produced after the second recovery and expansion can have a viability of 90% or more, and can be further expanded (such as third expansion and fourth expansion). The expanded cells can also be used for clinical treatment. Therefore, the method of the present disclosure greatly shortens the time for clinical patients to wait for Tregs, and can meet the clinical on-demand access requirement.

2. Flow cytometry results of cord blood-derived Tregs produced after the second expansion in the present disclosure show that, after the second expansion, a proportion of CD4⁺CD25⁺CD127⁻ cells is 99.6+0.15, and a proportion of CD3⁺CD8⁺ cells is less than 5%, indicating that a quality of the Tregs meets the standards of clinical research. Carboxyfluorescein diacetate succinimidyl ester (CFSE) assay results show that cord blood-derived Tregs produced after the second expansion still can significantly inhibit the proliferation ability of aggressive T cells.

### DESCRIPTION OF THE DRAWINGS

To describe the technical solutions in the embodiments of the present disclosure or in the prior art clearly, the accompanying drawings required for describing the embodiments or the prior art will be briefly described below. Apparently, the accompanying drawings in the following description show some embodiments of the present disclosure, and a person of ordinary skill in the art may still derive other accompanying drawings from these accompanying drawings without creative efforts.

FIG. 1 and FIG. 2 show the comparison of cell recovery viabilities of cryopreserved Tregs in different media (an X-VIVO medium, an expansion culture medium, and IL-2-containing expansion culture media (IL-2 treatment groups with different concentrations are set) and an expansion culture medium+magnetic beads;

FIG. 3 and FIG. 4 show the comparison of cell numbers of cryopreserved Tregs in different media (an X-VIVO medium, an expansion culture medium, and IL-2-containing expansion culture media (IL-2 treatment groups with different concentrations are set) and an expansion culture medium+magnetic beads;

FIG. 5 shows phenotypic detection results of cord blood-derived Tregs produced after second expansion in a preferred embodiment of the present disclosure; and

FIG. 6 shows inhibitory effects of expanded cord blood-derived Tregs on aggressive T cells (CD4+CD25−) of diabetes in a preferred embodiment of the present disclosure, where FIG. 6A shows an inhibitory effect of a negative control group; FIG. 6B shows an inhibitory effect of a positive control group; FIG. 6C shows an inhibitory effect of a combination of CD25-T cells and Tregs in 1:1; FIG. 6D shows an inhibitory effect of a combination of CD25-T cells and Tregs in 2:1; and FIG. 6E shows an inhibitory effect of a combination of CD25-T cells and Tregs in 4:1.

### SPECIFIC IMPLEMENTATIONS

In order to facilitate the understanding of the present disclosure, the present disclosure is described in detail below in conjunction with the accompanying drawings of the specification and the preferred embodiments, but the protection scope of the present disclosure is not limited to the following specific embodiments.

Unless otherwise defined, all technical terms used hereinafter have the same meaning as commonly understood by those skilled in the art. The technical terms used herein are merely for the purpose of describing specific embodiments, and are not intended to limit the protection scope of the present disclosure.

Unless otherwise specified, various raw materials, reagents, instruments, devices, etc. used in the present disclosure can be purchased from the market or can be prepared by the existing methods.

### EMBODIMENTS

A method for in vitro expansion of cryopreserved cord blood-derived Tregs with a high recovery rate was provided. With the method, Tregs could be subjected to recovery and expanded culture and then used in the preparation of a drug for treating an immune disease. The in vitro expansion method was as follows:

### I. Preparation of Cord Blood-Derived Tregs

1. Sterilization of a cord blood bag and preparation of cord blood-derived autologous plasma: Cord blood in the cord blood bag was transferred into a 50 mL centrifuge tube and centrifuged under the following conditions: 20° C., 3,000 rpm, 10 min, +8, and −9. A resulting supernatant was collected and transferred to a new 50 mL centrifuge tube and further centrifuged under the following conditions: 20° C., 4,000 rpm, 10 min, +8, and −9. Resulting yellow clear plasma was collected, inactivated in a 56° C. water bath for 30 min, and then placed in a refrigerator for later use. The remaining cord blood was resuspended with normal saline in the same volume as the collected plasma.

2. The cord blood resuspended in normal saline was slowly added to a lymphocyte separation solution (a volume of the cord blood: a volume of the lymphocyte separation solution=1:1), and centrifugation was conducted under the following conditions: 20° C., *400 rcf to *1610 rcf, 25 min, +0, and −0. After the centrifugation was completed, the cord blood was layered. A PBMC layer was slowly and gently collected with a pipette, where a lymphocyte liquid should be pipetted as little as possible.

3. The PBMC layer was further centrifuged under the following conditions: 20° C., *580 rcf, 10 min, +8, and −9. 5 mL to 10 mL of a 10× red blood cell lysis buffer and 45 mL of sterilized water were taken and prepared into a red blood cell lysis buffer. After the centrifugation was completed, a resulting supernatant was removed, and the red blood cell lysis buffer was added. 2 min to 10 min later, 0.9% normal saline was added to 35 mL to stop the red blood cell lysis. Centrifugation was conducted under the following conditions: 20° C., *290 rcf, 10 min, +8, and −9. A resulting supernatant was removed. The steps of adding 0.9% normal saline to a specified volume, centrifugation, and removing a resulting supernatant were repeated 2 times to 3 times.

4. After a supernatant produced after the final centrifugation was removed, resulting cells were loosened by flicking, 0.9% normal saline was added, and 10 μL of a resulting cell solution was taken for counting. 0.9% normal saline was added to the remaining cells to 40 mL. Centrifugation was further conducted under the following conditions: 20° C., *290 rcf, 10 min, +8, and −9. The counting was conducted through the following specific steps: The cell solution taken was added to an EP tube, 90 μL of trypan blue was added to the EP tube, and thorough mixing was conducted. 10 μL of a resulting mixture was taken and added to a hemacytometer, and quickly counted: (sum of numbers in four corners/4)×10⁵ cells/mL*dilution factor. For cells on lines, cells on the upper and left lines were counted, and cells on the lower and right lines were not counted.

5. After the centrifugation was completed, a resulting supernatant was removed, the residual supernatant could be removed by a pipette (1,000 μL) as much as possible, and cells were loosened by flicking. 3 mL of a cryopreservation solution was slowly added dropwise to the cells and gently pipetted up and down for thorough mixing. The remaining cryopreservation solution was added and pipetted up and down for thorough mixing. A resulting cell suspension was added to 5 mL freezing tubes at 4 mL/tube, programmed cooled to −80° C., and transferred to a liquid nitrogen tank. A preparation process of the cryopreservation solution was as follows: according to the cell number of 5×10⁶ to 10×10⁶/mL, a total of 40 mL of the cryopreservation solution was prepared with 90% of cord blood-derived autologous plasma and 10% of dimethyl sulfoxide (DMSO). During the preparation, the autologous plasma was added first and then DMSO was slowly added dropwise. DMSO was taken from bottom to top to prevent coagulation. The whole preparation process was conducted in the dark on ice.

6. A freezing tube with cryopreserved cells was taken out from the liquid nitrogen tank and immediately placed in a 37° C. water bath for recovery. The cells were resuspended with a serum-free medium in a 15 mL centrifuge tube, counted according to the method in the step 4, and then centrifuged under the following conditions: 20° C., *150 rcf to *390 rcf, 10 min, +8, and −9.

7. After the centrifugation was completed, a resulting supernatant was removed. 90 μL of MACS Buffer was added per 1×10⁷ cells/mL, that is, (n×90) μL. 10 μL to 20 μL of CD25 MicroBeadsII was added per 1×10⁷ cells/mL, that is, (n×10) μL. Vortexing was conducted.

8. Cells were resuspended with an Easy Buffer to produce 0.5 mL to 6 mL of a cell suspension with a cell concentration of 5×10⁷ cells/mL.

9. The cell suspension produced after the resuspending was transferred into a sterile tube matching with a magnetic pole.

10. A CD25-positive selection antibody was added at an amount of the resuspension volume×50 μL, and thorough mixing was conducted.

11. Incubation was conducted for 5 min.

12. Releasable spheres were vortexed for 30 s or more until magnetic bead aggregates disappeared.

13. The releasable spheres were added at an amount of the resuspension volume×30 μL, and thorough mixing was conducted.

14. A CD4⁺ T cell enrichment antibody mixture (T cell Enrichment Cocktail) was added at an amount of the resuspension volume×50 μL, and thorough mixing was conducted.

15. Incubation was conducted for 5 min.

16. An Easy Buffer was added to 10 mL, and gentle mixing was conducted 2 times to 3 times.

17. The sterile tube was placed on the magnetic pole without a cap.

18. Incubation was conducted for 10 min.

19. A centrifuge tube was prepared to collect CD25− cells, and a liquid in the sterile tube was poured into the centrifuge tube under the magnetic pole.

20. The sterile tube was removed from the magnetic pole, 10 mL of an Easy Buffer was added, and gentle mixing was conducted 2 times to 3 times. The sterile tube was placed on the magnetic pole, and incubation was conducted for 5 min.

21. The steps 19 and 20 were repeated twice.

22. An Easy Buffer was added to the initial resuspension volume with all cells on a tube wall rinsed off.

23. A magnetic bead-removing buffer (Release Buffer) was added at an amount of the resuspension volume×100 μL, and vigorous mixing was conducted 5 or more times.

24. A CD127high-removing antibody mixture (Depletion Cocktail) was added at an amount of the resuspension volume×50 μL, and thorough mixing was conducted.

25. Incubation was conducted for 5 min.

26. An Easy Buffer was added to 10 mL, and thorough mixing was conducted 2 times to 3 times.

27. The sterile tube was placed on the magnetic pole, and incubation was conducted for 5 min.

28. A centrifuge tube was prepared to collect Tregs, and a liquid in the sterile tube was poured into the prepared centrifuge tube under the magnetic pole to obtain the Tregs.

The above process for acquiring Tregs could adopt a EasySep™ human CD4⁺CD25⁺CD127− Treg sorting kit, with brand: STEMCELL Technologies and Item No.: 18063.

### II. Expansion of Tregs

1. Cells were inoculated into a 48-well plate at 2×10⁵ to 8×10⁵/mL, 0.5 mL of an expansion culture medium including magnetic beads (a number of the magnetic beads: a number of cells=1:1) was added per well on day 0 (a formula of the expansion culture medium was shown in Table 1), and the cells were cultured at 37° C. and 5% CO₂.

TABLE 1
Formula of 50 mL of the expansion culture medium for cord blood-derived Tregs

| | | | | Final | |
|---|---|---|---|---|---|
| Reagent name | Company | Item No. | Volume | concentration | |
| X-VIVO 15 w/o Gent or | Lonza | 04-744Q | 35.274 ml-42.69 ml | 70.54%-85.27% | |
| Phenol Red | | | | | |
| Hepes | Hyclone | SH3023701 | 1.25 ml-2.5 ml | 2.5%-5% | |
| PENICILLIN | Gibco | 15140122 | 0.5 ml-1 ml | 1%-2% | |
| STREPTOMYCIN | | | | | |
| L-Glutamine solution | sigma | G7513-100ML | 0.5 ml-1 ml | 1%-2% | |
| 1000X 2-Mercaptoethanol | Millipore | ES-007-E | 50 μl-100 μl | 50-100 | umol/L |
| rhIL-2 | R&D | 202-IL-010 | 7.69 μl-15.4 μl | 200-400 | U/ml |
| rapamycin | sigma | R8781-200UL | 1 μl-10 μl | 50-200 | nmol/L |

| | | | | |
|---|---|---|---|---|
| AB serum | Gemini | 100-512 | 5 ml-10 ml | 10%-20% |

2. On day 1 to day 2 after the inoculation (24 h to 48 h later), 0.5 mL to 1 mL of the expansion culture medium and 100 U/mL to 400 U/mL of rhIL-2 were added to cells in each well, and cells were cultured at 37° C. and 5% CO₂.

3. On day 3 to day 4 after the inoculation, a first passage was conducted as follows: 0.5 mL of the expansion culture medium was removed, the remaining expansion culture medium was thoroughly mixed with cells, and cells in each well were passaged to 2 wells. An expansion culture medium including 100 U/mL to 400 U/mL of rhIL-2 was added to 1 mL, and cells were cultured at 37° C. and 5% CO₂. After the addition, there was 1 mL of the expansion culture medium in each well, and the content of rhIL-2 added was based on 1 mL of the expansion culture medium. The amounts of rhIL-2 added in other steps were the same as the amount in this step.

4. On day 5 to day 6 after the inoculation, a second passage was conducted as follows: 0.5 mL of the expansion culture medium was removed, the remaining expansion culture medium was thoroughly mixed with cells, and cells in each well were passaged to 2 wells. An expansion culture medium including 100 U/mL to 400 U/mL of rhIL-2 was added to 1 mL, and cells were cultured at 37° C. and 5% CO₂.

5. On day 7 to day 10 after the inoculation, a third passage was conducted as follows: The anti-CD3CD28 magnetic beads in a Petri dish were removed, and then washed fresh anti-CD3CD28 magnetic beads (a number of the magnetic beads: a number of Tregs=1:1) were added. Cells were cultured at 37° C. and 5% CO₂.

6. On day 10 to day 11 after the inoculation, a fourth passage was conducted as follows: 0.5 mL of the expansion culture medium was removed, the remaining expansion culture medium was thoroughly mixed with cells, and cells in each well were passaged to 2 wells. An expansion culture medium including 100 U/mL to 400 U/mL of rhIL-2 was added to 1 mL, and cells were cultured at 37° C. and 5% CO₂.

7. On day 11 to day 12 after the inoculation, a fifth passage was conducted as follows: 0.5 mL of the expansion culture medium was removed, the remaining expansion culture medium was thoroughly mixed with cells, and cells in each well were passaged to 2 wells. An expansion culture medium including 100 U/mL to 400 U/mL of rhIL-2 was added to 1 mL, and cells were cultured at 37° C. and 5% CO₂.

8. On day 12 to day 13 after the inoculation, a sixth passage was conducted as follows: 0.5 mL of the expansion culture medium was removed, the remaining expansion culture medium was thoroughly mixed with cells, and cells in each well were passaged to 2 wells. An expansion culture medium including 100 U/mL to 400 U/mL of rhIL-2 was added to 1 mL, and cells were cultured at 37° C. and 5% CO₂.

9. On day 13 to day 14 after the inoculation, a seventh passage was conducted as follows: 0.5 mL of the expansion culture medium was removed, the remaining expansion culture medium was thoroughly mixed with cells, and cells in each well were passaged to 2 wells. An expansion culture medium including 100 U/mL to 400 U/mL of rhIL-2 was added to 1 mL, and cells were cultured at 37° C. and 5% CO₂.

10. On day 14 to day 16 after the inoculation, an eighth passage was conducted as follows: 0.5 mL of the expansion culture medium was removed, the remaining expansion culture medium was thoroughly mixed with cells, and cells in each well were passaged to 2 wells. An expansion culture medium including 100 U/mL to 400 U/mL of rhIL-2 was added to 1 mL, and cells were cultured at 37° C. and 5% CO₂.

11. On day 16 to day 18 after the inoculation, a ninth passage was conducted as follows: 0.5 mL of the expansion culture medium was removed, the remaining expansion culture medium was thoroughly mixed with cells, and cells in each well were passaged to 2 wells. An expansion culture medium including 100 U/mL to 400 U/mL of rhIL-2 was added to 1 mL, and cells were cultured at 37° C. and 5% CO₂.

In this embodiment, contents of components in the expansion culture medium for Tregs were expressed in volume fractions. A volume fraction refers to a volume proportion of each component. The expansion culture medium for Tregs included 70.54% to 85.27% of a serum-free medium, 2.5% to 5% of a 4-hydroxyethylpiperazine ethanesulfonic acid buffer, 1% to 2% of a penicillin-streptomycin solution, 1% to 2% of L-glutamine, 50 mol/L to 100 mol/L of 2-mercaptoethanol, 0 U/mL to 1,000 U/mL of recombinant IL-2, 50 nmol/L to 200 nmol/L of rapamycin, and 10% to 20% of AB serum.

The serum-free medium could be X-VIVO 15 w/o Gent or Phenol Red of Lonza, Item No.: 04-744Q. The 4-hydroxyethylpiperazine ethanesulfonic acid buffer could be a Hepes buffer of Hyclone, Item No.: SH3023701. The penicillin-streptomycin solution could be PENICILLIN STREPTOMYCIN of Gibco, 15140122. The L-glutamine could be an L-glutamine solution of sigma, Item No.: G7513-100ML. The 2-mercaptoethanol could be 1000X 2-Mercaptoethanol of Millipore, Item No.: ES-007-E. The recombinant IL-2 could be rhrhIL-2 of R&D, Item No.: 202-IL-010. The rapamycin could be rapamycin of sigma, Item No.: R8781-200UL. Of course, the same products produced by other companies could also be adopted. The AB serum was from Gemini, USA, Item No.: 100-512.

A preparation method of the above expansion culture medium slightly varied according to the specific use. For example, when Tregs were recovered and inoculated into a plate, the components of the expansion culture medium could be thoroughly mixed in advance and then directly added to a well plate, or the components other than the recombinant IL-2 for the expansion culture medium could be mixed to produce a premix and then the recombinant IL-2 was added. Because the recombinant IL-2 was basically exhausted in each well during the subculture, in the passage from 1 well to 2 wells, the premix was supplemented to each well. For example, 0.5 mL of the premix was supplemented to 1 mL of a solution in each well. In this case, the corresponding recombinant IL-2 should be added based on the amount of the expansion culture medium: 1 mL.

Criteria for the above expansion culture test were as follows:

Cord blood-derived Tregs to be recovered were divided into the following groups with a same cell number: an X-VIVO medium group (control), an expansion culture medium group, IL-2-containing expansion culture medium groups (IL-2 treatment groups with different concentrations: Treat 1: 10 U/mL to 75 U/mL; Treat 2: 50 U/mL to 150 U/mL; Treat 3: 100 U/mL to 250 U/mL; Treat 4: 200 U/mL to 600 U/mL; and Treat 5: 300 U/mL to 1,000 U/mL), and an expansion culture medium+magnetic bead group. Cells were inoculated into a 48-well plate at 1×10⁶ cells/well/mL for recovery. The cells were cultured for 48 h in an environment at 36° C. to 38° C. and 4% to 6% CO₂, and a cell viability and a cell number were measured. Then the cell expansion was conducted according to the established expansion conditions. A formula of the expansion culture medium was as follows: 42.69 parts of a serum-free medium, 1.25 parts of a 4-hydroxyethylpiperazine ethanesulfonic acid buffer, 0.5 part of a penicillin-streptomycin solution, 0.5 part of L-glutamine, 0.05 part of 2-mercaptoethanol, 0.01154 part of recombinant IL-2, 0.001 part of rapamycin, and 5 parts of AB serum.

There was a same initial number of Tregs for the groups, which was 1×10⁶. A number of Tregs in each group was counted. The counting was conducted through the following specific steps: 10 μL of a cell suspension was taken and added to an EP tube, 90 μL of trypan blue was added to the EP tube, and thorough mixing was conducted. 10 μL of a resulting mixture was taken and added to a hemacytometer, and quickly counted: (sum of numbers in four corners/4)×10⁵ cells/mL*dilution factor. For cells on lines, cells on the upper and left lines were counted, and cells on the lower and right lines were not counted. Living and dead cells were determined by trypan blue staining and flow cytometry, and blue-stained and positive cells were dead cells.

After the 48 h cell recovery, in the X-VIVO medium group, the expansion culture medium group, the IL-2-containing expansion culture medium groups (IL-2 treatment groups with different concentrations: 10 U to 1,000 U), and the expansion culture medium+magnetic bead group, viable cell proportions were as follows: Control: 72.54±12.6; Medium: 84.89±6.73; IL-2 treat 1: 91.96±6.57; IL-2 treat 2: 93.71±4.36; IL-2 treat 3: 90.62±11.44; IL-2 treat 4: 95.03±5.56; IL-2 treat 5: 94.93±3.6; and Beads: 86.85±9.5, respectively, and cell numbers (×10⁶) were as follows: Control: 0.56±0.11; Medium: 0.68±0.081; IL-2 treat 1: 0.94±0.26; IL-2 treat 2: 0.84±0.083; IL-2 treat 3: 0.92±0.197; IL-2 treat 4: 1.05±0.32; IL-2 treat 5: 0.91±0.21; and Beads: 0.4±0.187, respectively. The results indicated that the traditional direct magnetic bead stimulation method was not suitable for the expansion of cryopreserved Tregs, and the 48 h in vitro recovery with the IL-2-containing expansion culture medium could protect the viability of cells. It can be seen that the expansion culture medium of the present application could significantly promote the proliferation ability of cord blood-derived Tregs, and an appropriate concentration of IL-2 was 200 U/mL to 600 U/mL.

A total number of Tregs in the IL-2-containing expansion culture medium group was 3×10⁷ on day 21 of expansion. Results of flow cytometry-based phenotypic and functional assays showed that the expanded Tregs had an immunosuppressive ability and met the standard of clinical research.

Experiment 1: A Phenotype of Cord Blood-Derived Tregs was Stable after Expansion

1. One well of Tregs (5×10⁵ to 10×10⁵) were taken, and 0.9% normal saline was added to 5 mL (room temperature). Centrifugation was conducted under the following conditions: 20° C., *570 rcf, 5 min, +8, and −9.

2. A phenotype of Tregs was detected with a PerFix-nc Kit (Item No.: PN B10825, Beckman). This kit included the following three buffers: buffer 1: Fixative Reagent, PN B10827-75tests-liquid; buffer 2: Permeabilizing Reagent, PN B10828-75tests-liquid; and buffer 3: Final 10×Solution, PN B10829-75tests-liquid. A supernatant produced after the centrifugation was removed by a pipette tip with 50 μL to 100 μL of a liquid left, and then 5 μL of the buffer 1 and 5 μL of an Fc Block antibody were added. Shaking was conducted (moderately), and incubation was conducted at room temperature in the dark for 15 min.

3. 5 μL of each of CD4, CD25, and CD127 antibodies was added. Shaking was conducted (moderately), and incubation was conducted at room temperature in the dark for 15 min to 30 min.

4. 3 mL of the buffer 3 diluted into a working solution (buffer 3: water=1:9) was added. Centrifugation was conducted under the following conditions: 20° C., *570 rcf, 5 min, +8, and −9.

5. Flow cytometry was conducted. Flow cytometry results showed that, on day 18 of expansion, a proportion of CD4⁺CD25⁺CD127⁻ cells was 97.07+2.03 and a proportion of CD3+CD8+ cells was 4.69+2.0, indicating that a quality of the Tregs reached the standard of clinical research.

Experiment 2: Cord Blood-Derived Tregs could Significantly Inhibit the Proliferation Ability of Aggressive T Cells after Expansion

1. CD4(+)CD25(−) aggressive T cells were recovered, and 1×10⁶ cells were taken.

2. The cells (1×10⁶ cells) were resuspended with 1 mL of a serum-free medium.

3. 1 μL of a cell trace stock solution was added per 1 mL of a cell suspension (CellTrace™ CFSE Cell Proliferation Kit, Item No.: C34554, Thermofisher scientific. This kit included 10 parts of single-use bottled CellTrace™ CFSE (component A) and 1 part of bottled DMSO (component B). The cell trace stock solution was a mixed solution of 18 μL of DMSO and one part of bottled CellTrace™ CFSE prepared according the instructions of the kit for CFSE staining of cells. Details could be seen on https://www.thermofisher.com/order/catalog/product/C34554).

4. Incubation was conducted for 20 min at 37° C. in the dark.

5. 5 mL of a washing solution (serum-free medium+10% fetal bovine serum (FBS) was added.

6. Incubation was conducted for 5 min at 37° C. in the dark.

7. Centrifugation was conducted under the following conditions: 20° C., *290 rcf, 10 min, +8, and −9.

8. Cells were resuspended with a medium (serum-free medium+10% FBS) pre-warmed at 37° C.

9. One well of Tregs were taken, subjected to magnetic bead removal, counted, and centrifuged under the following conditions: 20° C., *290 rcf, 10 min, +8, and −9.

10. 1×10⁶ Tregs were resuspended with a medium (serum-free medium+10% FBS) pre-warmed at 37° C.

11. CD4(+)CD25− T cells and Tregs were co-cultured in a 96-well plate with a number ratio of the CD4(+)CD25− T cells to the Tregs being 1:1, 2:1, and 4:1 (in each well, there was 200 μL in total, and 3 μL of magnetic beads was added). A CD4(+)CD25− T cell group treated with CFSE alone was set as a negative control group, and a CD4(+)CD25− T cell group treated with CFSE (magnetic bead stimulation) was set as a positive control group.

12. An inhibitory function of cord blood-derived Tregs was detected by flow cytometry.

13. Results of the CFSE experiment showed that, when CD4(+) CD25− T cells and Tregs were co-cultured in number ratios of 1, 2, and 4:1, inhibitory rates for the proliferation of CD4(+) CD25− T cells were 77.27±15.5%, 74.53±18.36%, 74±17.1%, and 72.34±14.1%, respectively. The results indicated that the expanded cord blood-derived Tregs could significantly inhibit the proliferation ability of aggressive T cells.

### III. Cryopreservation and Recovery of Expanded Tregs

1. Tregs produced after 14 d to 21 d of expansion were collected in a centrifuge tube with an expansion culture medium. Centrifugation was conducted, a resulting supernatant was removed, and resulting cells were loosened by flicking. 0.9% normal saline was added, and 10 μL of a resulting cell solution was taken for counting. 0.9% normal saline was added to the remaining cells to 40 mL. Centrifugation was further conducted under the following conditions: 20° C., *290 rcf, 10 min, +8, and −9. The counting was conducted through the following specific steps: The cell solution taken was added to an EP tube, 90 μL of trypan blue was added to the EP tube, and thorough mixing was conducted. 10 μL of a resulting mixture was taken and added to a hemacytometer, and quickly counted: (sum of numbers in four corners/4)×10⁵ cells/mL*dilution factor. For cells on lines, cells on the upper and left lines were counted, and cells on the lower and right lines were not counted.

2. After the centrifugation was completed, a resulting supernatant was removed, the residual supernatant could be removed by a pipette (1,000 μL) as much as possible, and cells were loosened by flicking. 3 mL of a cryopreservation solution was slowly added dropwise to the cells and gently pipetted up and down for thorough mixing. The remaining cryopreservation solution was added and pipetted up and down for thorough mixing. A resulting cell suspension was added to 5 mL freezing tubes at 4 mL/tube, programmed cooled to −80° C., and transferred to a liquid nitrogen tank. A preparation process of the cryopreservation solution was as follows: according to the cell number of 5×10⁶ to 10×10⁶/mL, a total of 40 mL of the cryopreservation solution was prepared with 90% of AB serum and 10% of DMSO. During the preparation, the autologous plasma was added first and then DMSO was slowly added dropwise. DMSO was taken from bottom to top to prevent coagulation. The whole preparation process was conducted in the dark on ice.

3. A freezing tube with cryopreserved cells was taken out from the liquid nitrogen tank and immediately placed in a 37° C. water bath for recovery. The cells were resuspended with a serum-free medium in a 15 mL centrifuge tube, counted according to the method in the step 1, and then centrifuged under the following conditions: 20° C., *150 rcf to *390 rcf, 10 min, +8, and −9.

4. Cells were inoculated into a 48-well plate at 2×10⁵ to 8×10⁵/mL, 0.5 mL of an expansion culture medium including 10 U/mL to 2,000 U/mL of rhIL-2 was added per well on day 0, and the cells were cultured at 37° C. and 5% CO₂.

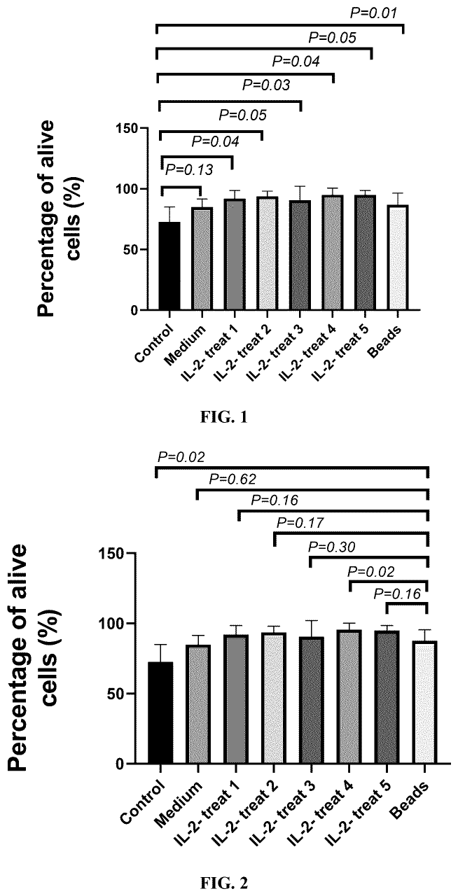

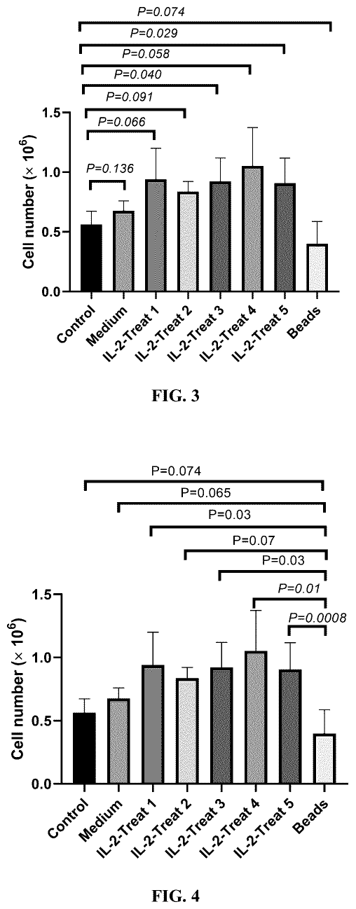

5. On day 1 to day 3 after inoculation (24 h to 72 h later), a viability and number of cells were detected with trypan blue (a detailed method could be seen in the step 4 of “I. Preparation of cord blood-derived Tregs”), as shown in FIG. 1 to FIG. 4. 0.5 mL to 1 mL of an expansion culture medium, magnetic beads (a number of the magnetic beads: a number of cells=1:1), and 100 U/mL to 400 U/mL of rhIL-2 were added to cells in each well, and cells were cultured at 37° C. and 5% CO₂.

6. On day 3 to day 4 after the inoculation, a first passage was conducted as follows: 0.5 mL of the expansion culture medium was removed, the remaining expansion culture medium was thoroughly mixed with cells, and cells in each well were passaged to 2 wells. An expansion culture medium including 100 U/mL to 400 U/mL of rhIL-2 was added to 1 mL, and cells were cultured at 37° C. and 5% CO₂. After the addition, there was 1 mL of the expansion culture medium in each well, and the content of rhIL-2 added was based on 1 mL of the expansion culture medium. The amounts of rhIL-2 added in other steps were the same as the amount in this step.

7. On day 5 to day 6 after the inoculation, a second passage was conducted as follows: 0.5 mL of the expansion culture medium was removed, the remaining expansion culture medium was thoroughly mixed with cells, and cells in each well were passaged to 2 wells. An expansion culture medium including 100 U/mL to 400 U/mL of rhIL-2 was added to 1 mL, and cells were cultured at 37° C. and 5% CO₂.

8. On day 7 to day 10 after the inoculation, a third passage was conducted as follows: The anti-CD3CD28 magnetic beads in a Petri dish were removed, and then washed fresh anti-CD3CD28 magnetic beads (a number of the magnetic beads: a number of Tregs=1:1) were added. Cells were cultured at 37° C. and 5% CO₂.

9. On day 10 to day 11 after the inoculation, a fourth passage was conducted as follows: 0.5 mL of the expansion culture medium was removed, the remaining expansion culture medium was thoroughly mixed with cells, and cells in each well were passaged to 2 wells. An expansion culture medium including 100 U/mL to 1,000 U/mL of rhIL-2 was added to 1 mL, and cells were cultured at 37° C. and 5% CO₂.

10. On day 11 to day 12 after the inoculation, a fifth passage was conducted as follows: 0.5 mL of the expansion culture medium was removed, the remaining expansion culture medium was thoroughly mixed with cells, and cells in each well were passaged to 2 wells. An expansion culture medium including 100 U/mL to 1,000 U/mL of rhIL-2 was added to 1 mL, and cells were cultured at 37° C. and 5% CO₂.

11. On day 12 to day 13 after the inoculation, a sixth passage was conducted as follows: 0.5 mL of the expansion culture medium was removed, the remaining expansion culture medium was thoroughly mixed with cells, and cells in each well were passaged to 2 wells. An expansion culture medium including 100 U/mL to 1,000 U/mL of rhIL-2 was added to 1 mL, and cells were cultured at 37° C. and 5% CO₂.

12. On day 13 to day 14 after the inoculation, a seventh passage was conducted as follows: 0.5 mL of the expansion culture medium was removed, the remaining expansion culture medium was thoroughly mixed with cells, and cells in each well were passaged to 2 wells. An expansion culture medium including 100 U/mL to 1,000 U/mL of rhIL-2 was added to 1 mL, and cells were cultured at 37° C. and 5% CO₂.

Experiment 3: A Phenotype of Cord Blood-Derived Tregs was Stable after the Second Expansion

1. One well of Tregs (5×10⁵ to 10×10⁵) were taken, and 0.9% normal saline was added to 5 mL (room temperature). Centrifugation was conducted under the following conditions: 20° C., *570 rcf, 5 min, +8, and −9.

2. A phenotype of Tregs was detected with a PerFix-nc Kit (Item No.: PN B10825, Beckman). This kit included the following three buffers: buffer 1: Fixative Reagent, PN B10827-75tests-liquid; buffer 2: Permeabilizing Reagent, PN B10828-75tests-liquid; and buffer 3: Final 10×Solution, PN B10829-75tests-liquid. A supernatant produced after the centrifugation was removed by a pipette tip with 50 μL to 100 μL of a liquid left, and then 5 μL of the buffer 1 and 5 μL of an Fc Block antibody were added. Shaking was conducted (moderately), and incubation was conducted at room temperature in the dark for 15 min.

3. 5 μL of each of CD4, CD25, and CD127 antibodies was added. Shaking was conducted (moderately), and incubation was conducted at room temperature in the dark for 15 min to 30 min.

4. 3 mL of the buffer 3 diluted into a working solution (buffer 3: water=1:9) was added. Centrifugation was conducted under the following conditions: 20° C., *570 rcf, 5 min, +8, and −9.

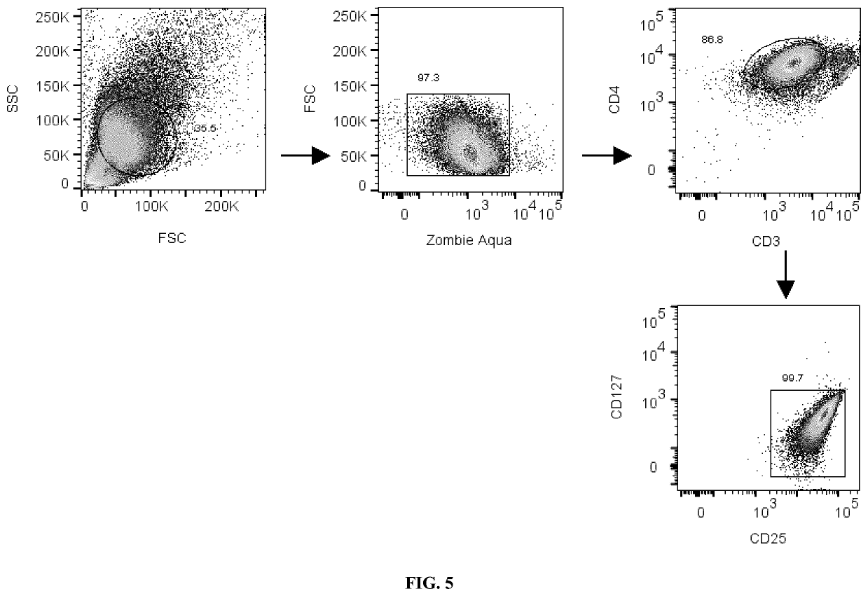

5. Flow cytometry was conducted. Flow cytometry results showed that, after the expansion, a proportion of CD4⁺CD25⁺CD127⁻ cells was 99.6+0.15 and a proportion of CD3⁺CD8⁺ cells was less than 5%, indicating that a quality of the Tregs reached the standard of clinical research. Test results were shown in FIG. 5.

Experiment 4: Cord Blood-Derived Tregs could Significantly Inhibit the Proliferation Ability of Aggressive T Cells after the Second Expansion

1. CD4(+)CD25(−) aggressive T cells were recovered, and 1×10⁶ cells were taken.

2. The cells (1×10⁶ cells) were resuspended with 1 mL of a serum-free medium.

3. 1 μL of a cell trace stock solution was added per 1 mL of a cell suspension (CellTrace™ CFSE Cell Proliferation Kit, Item No.: C34554, Thermofisher scientific. This kit included 10 parts of single-use bottled CellTrace™ CFSE (component A) and 1 part of bottled DMSO (component B). The cell trace stock solution was a mixed solution of 18 μL of DMSO and one part of bottled CellTrace™ CFSE prepared according the instructions of the kit for CFSE staining of cells. Details could be seen on https://www.thermofisher.com/order/catalog/product/C34554).

4. Incubation was conducted for 20 min at 37° C. in the dark.

5. 5 mL of a washing solution (serum-free medium+10% fetal bovine serum (FBS) was added.

6. Incubation was conducted for 5 min at 37° C. in the dark.

7. Centrifugation was conducted under the following conditions: 20° C., *290 rcf, 10 min, +8, and −9.

8. Cells were resuspended with a medium (serum-free medium+10% FBS) pre-warmed at 37° C.

9. One well of Tregs were taken, subjected to magnetic bead removal, counted, and centrifuged under the following conditions: 20° C., *290 rcf, 10 min, +8, and −9.

10. 1×10⁶ Tregs were resuspended with a medium (serum-free medium+10% FBS) pre-warmed at 37° C.

11. CD4(+)CD25− T cells and Tregs were co-cultured in a 96-well plate with a number ratio of the CD4(+)CD25− T cells to the Tregs being 1:1, 2:1, and 4:1 (in each well, there was 200 μL in total, and 3 μL of magnetic beads was added). A CD4(+)CD25− T cell group treated with CFSE alone was set as a negative control group, and a CD4(+)CD25− T cell group treated with CFSE (magnetic bead stimulation) was set as a positive control group.

12. An inhibitory function of cord blood-derived Tregs was detected by flow cytometry.

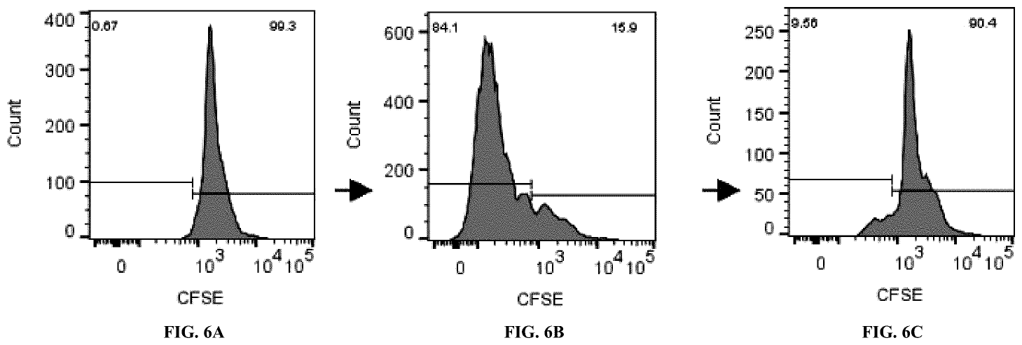

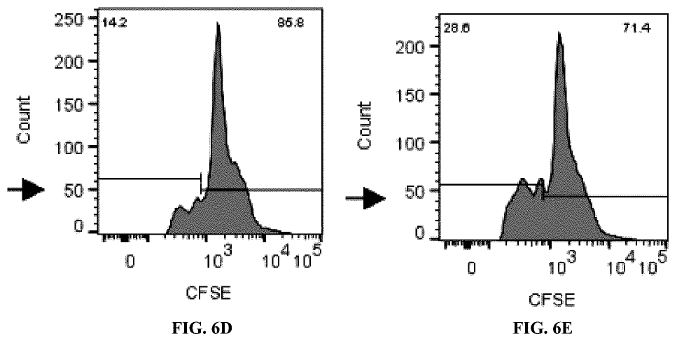

13. Results of the CFSE experiment showed that, when CD4(+) CD25-T cells and Tregs were co-cultured in number ratios of 1, 2, and 4:1, inhibitory rates for the proliferation of CD4H CD25⁻ T cells were 91.36±4.1%, 83.2±7.4%, and 63±17.4%, respectively. The results indicated that the cord blood-derived Tregs produced after the second expansion could significantly inhibit the proliferation ability of aggressive T cells, as shown in FIG. 6.

What is claimed is:

1-10. (canceled)

11. A method for in vitro expansion of cryopreserved cord blood-derived regulatory T cells (Tregs) with a high recovery rate, comprising the following steps:
(1) preparing Tregs, adding an expansion culture medium to Tregs carrying magnetic beads, and conducting a first expansion culture, wherein a primary culture is conducted for 1 d to 2 d, then a subculture is conducted once every 1 d to 3 d, and a total culture time is 18 d or more; recovering the cryopreserved cord blood-derived Tregs after the first expansion culture, and resuspending cells with a serum-free medium to produce a suspension; wherein
a process for the preparing Tregs comprises the following steps:
S1, isolating a peripheral blood mononuclear cell (PBMC) layer from cord blood, removing red blood cells with a red blood cell lysis buffer, washing, and counting; conducting centrifugation, removing a first resulting supernatant, and cryopreserving; and recovering and counting;
S2, conducting centrifugation, removing a second resulting supernatant, and resuspending cells with an Easy Buffer to produce 0.5 mL to 6 mL of a cord blood-derived Treg-containing suspension with a cell concentration of 5×10⁷ cells/mL; and
S3, transferring the cord blood-derived Treg-containing suspension into a sterile tube matching with a magnetic pole, adding CD25 Positive Selection Cocktail, and incubating for 5 min; vortexing Releasable Rapidsphere for 30 s or more until magnetic bead aggregates disappear; adding the Releasable Rapidsphere; adding a CD4⁺T cell enrichment antibody mixture, and incubating for 5 min; adding the Easy Buffer to 10 mL, and gently mixing 2 times to 3 times; placing the sterile tube on the magnetic pole, and incubating for 10 min; preparing a first centrifuge tube to collect CD25⁻ cells, and pouring a first liquid in the sterile tube into the first centrifuge tube under the magnetic pole; removing the sterile tube from the magnetic pole, adding 10 mL of the Easy Buffer to the sterile tube, gently mixing 2 times to 3 times, placing the sterile tube on the magnetic pole, and incubating for 5 min; adding the Easy Buffer to an initial resuspension volume with all cells on a tube wall rinsed off, adding a magnetic bead-removing buffer; adding a CD127^{high}-removing antibody mixture, and incubating for 5 min; adding the Easy Buffer to 10 mL, and thoroughly mixing 2 times to 3 times; placing the sterile tube on the magnetic pole, and incubating for 5 min; and preparing a second centrifuge tube to collect the Tregs, and pouring a second liquid in the sterile tube into the second centrifuge tube under the magnetic pole to obtain the Tregs; wherein a process for obtaining the Tregs adopts a EasySep™ human CD4⁺CD25⁺CD127⁻ Treg sorting kit, with brand: STEMCELL Technologies and Item No.: 18063;
a process for the first expansion culture comprises the following steps:
inoculating cells into a 48-well plate at 2×10⁵ to 8×10⁵/mL, adding 0.5 mL of an expansion culture medium comprising magnetic beads per well on day 0, and culturing at 37° C. and 5% CO₂, wherein a number of the magnetic beads: a number of the cells=1:1;
on day 1 to day 2 after the inoculation, adding 0.5 mL to 1 mL of the expansion culture medium and 100 U/mL to 400 U/mL of rhIL-2 to cells in each well, and culturing at 37° C. and 5% CO₂;
on day 3 to day 4 after the inoculation, a first passage is conducted as follows: 0.5 mL of the expansion culture medium is removed, a remaining expansion culture medium is thoroughly mixed with cells, and cells in each well are passaged to 2 wells, an expansion culture medium comprising 100 U/mL to 400 U/mL of rhIL-2 is added to 1 mL, and the cells are cultured at 37° C. and 5% CO₂; after the addition, 1 mL of the expansion culture medium is presented in each well, and an amount of rhIL-2 added is based on 1 mL of the expansion culture medium, amounts of rhIL-2 added in other steps are equal to the amount in this step;
on day 5 to day 6 after the inoculation, a second passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 400 U/mL of rhIL-2 is added to 1 mL, and the cells are cultured at 37° C. and 5% CO₂;
on day 7 to day 10 after the inoculation, a third passage is conducted as follows: anti-CD3CD28 magnetic beads in a Petri dish are removed, and then washed fresh anti-CD3CD28 magnetic beads are added according to a number of the magnetic beads: a number of the Tregs=1:1, and cells are cultured at 37° C. and 5% CO₂;
on day 10 to day 11 after the inoculation, a fourth passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 400 U/mL of rhIL-2 is added to 1 mL, and the cells are cultured at 37° C. and 5% CO₂;
on day 11 to day 12 after the inoculation, a fifth passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 400 U/mL of rhIL-2 is added to 1 mL, and the cells are cultured at 37° C. and 5% CO₂;
on day 12 to day 13 after the inoculation, a sixth passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 400 U/mL of rhIL-2 is added to 1 mL, and the cells are cultured at 37° C. and 5% CO₂;
on day 13 to day 14 after the inoculation, a seventh passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 400 U/mL of rhIL-2 is added to 1 mL, and the cells are cultured at 37° C. and 5% CO₂;
on day 14 to day 16 after the inoculation, an eighth passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 400 U/mL of rhIL-2 is added to 1 mL, and the cells are cultured at 37° C. and 5% CO₂; and
on day 16 to day 18 after the inoculation, a ninth passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 400 U/mL of rhIL-2 is added to 1 mL, and the cells are cultured at 37° C. and 5% CO₂;
(2) adding a recovered suspension to the expansion culture medium, and conducting a second expansion culture, wherein a primary culture is conducted for 1 d to 2 d, then a subculture is conducted once every 1 d to 3 d, and a total culture time is 13 d or more; wherein
a process for the second expansion culture comprises the following steps:
inoculating cells into a 48-well plate at 2×10⁵ to 8×10⁵/mL, adding 0.5 mL of an expansion culture medium comprising 10 U/mL to 2000 U/mL of rhIL-2 per well on day 0, and culturing at 37° C. and 5% CO₂;
on day 1 to day 3 after the inoculation, detecting a viability and number of cells with trypan blue, adding 0.5 mL to 1 mL of the expansion culture medium, the magnetic beads, and 100 U/mL to 400 U/mL of rhIL-2 to cells in each well, and culturing at 37° C. and 5% CO₂, wherein a number of the magnetic beads: a number of the cells=1:1;
on day 3 to day 4 after the inoculation, a first passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 400 U/mL of rhIL-2 is added to 1 mL, and the cells are cultured at 37° C. and 5% CO₂; after the addition, 1 mL of the expansion culture medium is presented in each well, and an amount of rhIL-2 added is based on 1 mL of the expansion culture medium, amounts of rhIL-2 added in other steps are equal to the amount in this step;
on day 5 to day 6 after the inoculation, a second passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 400 U/mL of rhIL-2 is added to 1 mL, and the cells are cultured at 37° C. and 5% CO₂;
on day 7 to day 10 after the inoculation, a third passage is conducted as follows: anti-CD3CD28 magnetic beads in a Petri dish are removed, and then washed fresh anti-CD3CD28 magnetic beads are added according to a number of the magnetic beads: a number of the Tregs=1:1, and cells are cultured at 37° C. and 5% CO₂;
on day 10 to day 11 after the inoculation, a fourth passage is conducted as follows: 0.5 mL of the expansion culture medium is removed, a remaining expansion culture medium is thoroughly mixed with cells, and cells in each well are passaged to 2 wells, an expansion culture medium comprising 100 U/mL to 1000 U/mL of rhIL-2 is added to 1 mL, and the cells are cultured at 37° C. and 5% CO₂;
on day 11 to day 12 after the inoculation, a fifth passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 1000 U/mL of rhIL-2 is added to 1 mL, and the cells are cultured at 37° C. and 5% CO₂;
on day 12 to day 13 after the inoculation, a sixth passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 1000 U/mL of rhIL-2 is added to 1 mL, and the cells are cultured at 37° C. and 5% CO₂; and
on day 13 to day 14 after the inoculation, a seventh passage is conducted as follows: 0.5 mL of the expansion culture medium is removed, a remaining expansion culture medium is thoroughly mixed with cells, and cells in each well are passaged to 2 wells, the expansion culture medium comprising 100 U/mL to 1000 U/mL of rhIL-2 is added to 1 mL, and the cells are cultured at 37° C. and 5% CO₂;
wherein, according to a final concentration, the expansion culture medium comprises 70.54% to 85.27% volume fraction of the serum-free medium, 2.5% to 5% volume fraction of a 4-hydroxyethylpiperazine ethanesulfonic acid buffer, 1% to 2% volume fraction of a penicillin-streptomycin solution, 1% to 2% volume fraction of L-glutamine, 50 μmol/L to 100 μmol/L of 2-mercaptoethanol, 200 U/mL to 400 U/mL of recombinant interleukin-2 (IL-2), 50 nmol/L to 200 nmol/L of rapamycin, and 10% to 20% volume fraction of AB serum;
the magnetic beads used in the first expansion culture and the second expansion culture are both the anti-CD3CD28 magnetic beads.

12. The method for the in vitro expansion of the cryopreserved cord blood-derived Tregs with the high recovery rate according to claim 11, wherein an amount of the CD25 Positive Selection Cocktail added is a resuspension volume×50 μL, an amount of the Releasable Rapidsphere added is the resuspension volume×30 μL, an amount of the CD4⁺T cell enrichment antibody mixture added is the resuspension volume×50 μL, an amount of the magnetic bead-removing buffer added is the resuspension volume×100 μL, and an amount of the CD127^{high}-removing antibody mixture added is the resuspension volume×50 μL.
